(19) United States
(12) Patent Application Publication
(10) Pub. No.: US 2012/0308626 A1
(43) Pub. Date: Dec. 6, 2012

(54) COMPOSITION FOR CLEANING TEETH COMPRISING NATURAL GLASS AND RELATED METHODS

(75) Inventor: Bo Wang, Lompoc, CA (US)

(73) Assignee: Imerys Filtration Minerals, Inc., San Jose, CA (US)

(21) Appl. No.: 13/577,711

(22) PCT Filed: Jan. 25, 2011
(86) PCT No.: PCT/US11/22317

§ 371 Date: Aug. 8, 2012

Related U.S. Application Data
(60) Provisional application No. 61/303,487, filed on Feb. 11, 2010.

(51) Int. Cl. A61K 8/19; C03C 12/00; A61K 8/02

(52) U.S. Cl. 424/401; 424/49; 428/402

(57) ABSTRACT

A composition for cleaning teeth may include natural glass, wherein the natural glass has a top particle size (d₉₀) less than 50 μm and a median particle size (d₅₀) less than 30 μm, and wherein the natural glass ranges from 0.1 percent to 20 percent by weight of the composition. A natural glass composition may have a top particle size (d₉₀) less than 50 μm, a median particle size (d₅₀) less than 30 μm, and exhibit an RDA value less than 220. The composition for cleaning teeth may include a toothpaste base.

### CLAIM OF PRIORITY

This PCT International Application claims the benefit of priority of U.S. Provisional Patent Application No. 61/303,487, filed Feb. 11, 2010, the subject matter of which is incorporated herein by reference in its entirety.

### FIELD OF THE DISCLOSURE

The present disclosure relates to compositions for cleaning teeth including natural glass and related methods. In particular, the present disclosure relates to compositions for cleaning teeth including natural glass having a defined particle size characteristic, and related methods.

### BACKGROUND OF THE DISCLOSURE

Dental care includes the use of compositions for cleaning teeth, such as, for example, dentifrice compositions such as pastes or powders for cleaning teeth. Toothpaste is a commonly known example of a dentifrice composition, which typically has a paste-like form and which may include one or more components, such as, for example, binders, humectants, abrasives, detergents, flavoring agents, and preventatives, such as anti-infective agents and/or other medicaments.

The abrasive component in toothpaste serves to improve its cleaning effectiveness. However, while abrasives may improve cleaning effectiveness, they may also lead to undesirable erosion of the teeth.

One composition that has been added to toothpaste to improve its cleaning effectiveness is perlite. Perlite is an example of a naturally-occurring glass, such as, for example, an amorphous volcanic glass having a relatively high water content. By virtue of its relatively high water content, perlite expands when heated, for example above about 850-900° C. While perlite may improve the cleaning effectiveness of toothpaste, it may also lead to premature degradation of teeth due to its inherently abrasive nature.

Thus, it may be desirable to provide compositions that assist with the effectiveness of cleaning teeth, but which do not lead to premature erosion of the teeth due to excessive abrasiveness.

### SUMMARY

In the following description, certain aspects and embodiments will become evident. It should be understood that the aspects and embodiments, in their broadest sense, could be practiced without having one or more features of these aspects and embodiments. Thus, it should be understood that these aspects and embodiments are merely exemplary.

One aspect of the disclosure relates to a composition for cleaning teeth. The composition may include natural glass, wherein the natural glass has a top particle size (d₉₀) less than 50 micrometers (μm) and a median particle size (d₅₀) less than 30 μm. The natural glass may range from 0.1 percent to 20 percent by weight of the composition. Top particle size (d₉₀) is defined as the size for which 90 percent of the volume of the particles is smaller than the indicated size. Median particle size (d₅₀) is defined as the size for which 50 percent of the volume of the particles is smaller than the indicated size.

According to a further aspect, a natural glass may have a top particle size (d₉₀) less than 50 μm, a median particle size (d₅₀) less than 30 μm, and exhibit a relative dentin abrasion (RDA) value less than 200. RDA testing is a method of measuring of the erosive effect on tooth dentin of abrasives in compositions for cleaning teeth, and RDA value is standardized in accordance with DIN/ISO standard 11609, a standard that has been adopted by the American Dental Association (ADA). Higher RDA values indicate higher levels of abrasiveness.

According to another aspect, the natural glass may include perlite. For example, the natural glass may include expanded perlite, for example, milled, expanded perlite. According to some aspects, the perlite may include unexpanded perlite.

According to still another aspect, the glass may be selected from pumice, obsidian, volcanic ash, and shirasu.

According to yet a further aspect, the natural glass may have a top particle size (d₉₀) less than 40 μm, for example, a top particle size (d₉₀) less than 35 μm. According to some aspects, the natural glass may have a top particle size (d₉₀) ranging from 30 μm to 40 μm.

According to yet another aspect, the natural glass may have a median particle size (d₅₀) less than 25 μm, for example, a median particle size (d₅₀) ranging from 15 μm to 25 μm.

According still another aspect, the natural glass may range from 0.1 percent to 25 percent by weight of the composition, or, for example, from 0.1 percent to 10 percent by weight of the composition.

In another aspect, the natural glass can have a blue light brightness of greater than 70. In another aspect, the natural glass can have a whiteness (L-value) of greater than 75, such as, for example, greater than 80. In yet another aspect, the natural glass can have a yellowness (b-value) of less than 5, such as, for example, less than 2. In yet another aspect, the natural glass can have a redness (a-value) of less than 1.0, such as, for example, less than 0.5.

According to still a further aspect, the composition may exhibit an RDA value less than 230, for example, an RDA value less than 200. According to a further aspect, the composition may exhibit a pellicle cleaning ratio (PCR) value of at least 110, for example, a PCR value of at least 120. The PCR test is a laboratory method accepted by the ADA as useful in characterizing the stain cleaning actions of compositions for cleaning teeth. Test data are referenced against that of the ADA's reference material, calcium pyrophosphate, with the stain reduction resulting from calcium pyrophosphate being taken to be by definition 100. Higher values of PCR indicate greater stain removal or “whitening.” According to a further aspect, the composition may exhibit tooth polishing effect.

According yet another aspect, the composition may include a toothpaste base. For example, the toothpaste base may include at least one of binders (thickeners), humectants, abrasives, detergents, flavoring agents, and preventatives. According to still a further aspect, the natural glass may exhibit toothpaste thickening effect.

Additional objects and advantages of the disclosure will be set forth in part in the description which follows, and in part will be obvious from the description, or may be learned by practice of the disclosed embodiments.

Aside from the arrangements set forth above, the embodiments could include a number of other arrangements, such as those explained hereinafter. It is to be understood that both the foregoing description and the following description are exemplary only.

### BRIEF DESCRIPTION OF THE DRAWINGS

The accompanying drawings, which are incorporated in and constitute a part of this description, illustrate several exemplary embodiments and together with the description, serve to explain the principles of the embodiments. In the drawings,

FIG. 1 is a scanning electron micrograph of coarse fraction of a classified, expanded perlite.

FIG. 2 is a scanning electron micrograph of fine fraction of a classified, expanded perlite sample according to an exemplary embodiment;

FIG. 3 is a graph showing Relative Dentin Abrasion (RDA) test results for three examples of natural glass vs. top particle size (d₉₀); and

FIG. 4 is a graph showing Pellicle Cleaning Ratio (PCR) test results for three examples of natural glass vs. top particle size (d₉₀).

### DESCRIPTION OF EXEMPLARY EMBODIMENTS

Reference will now be made in more detail to a number of exemplary embodiments of the invention.

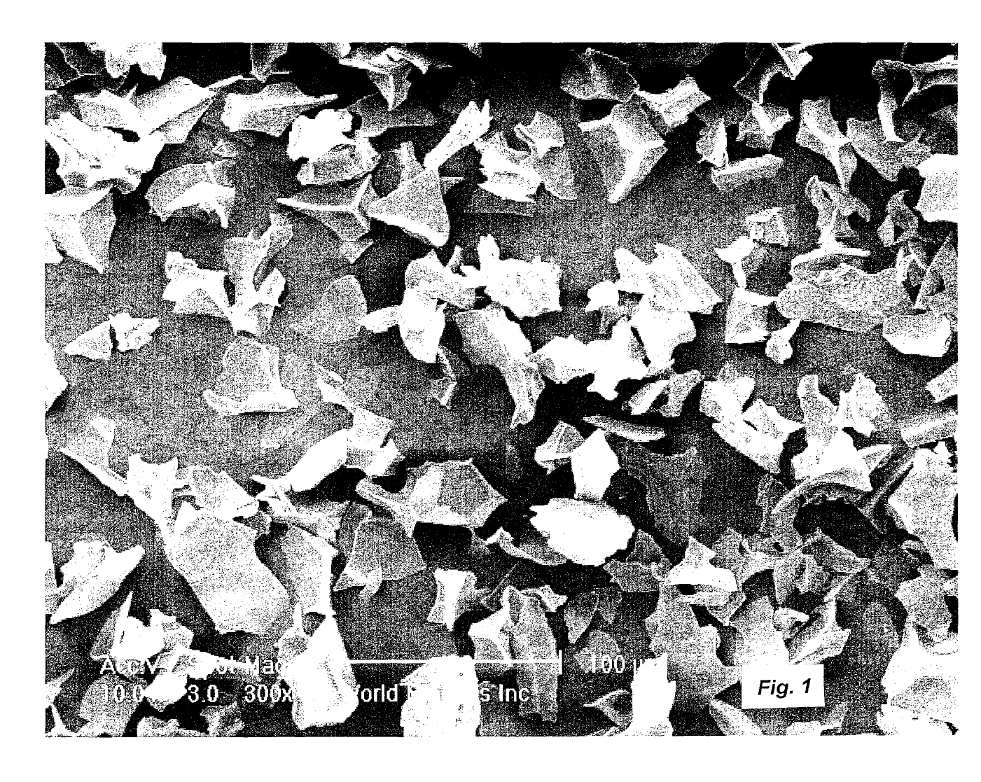

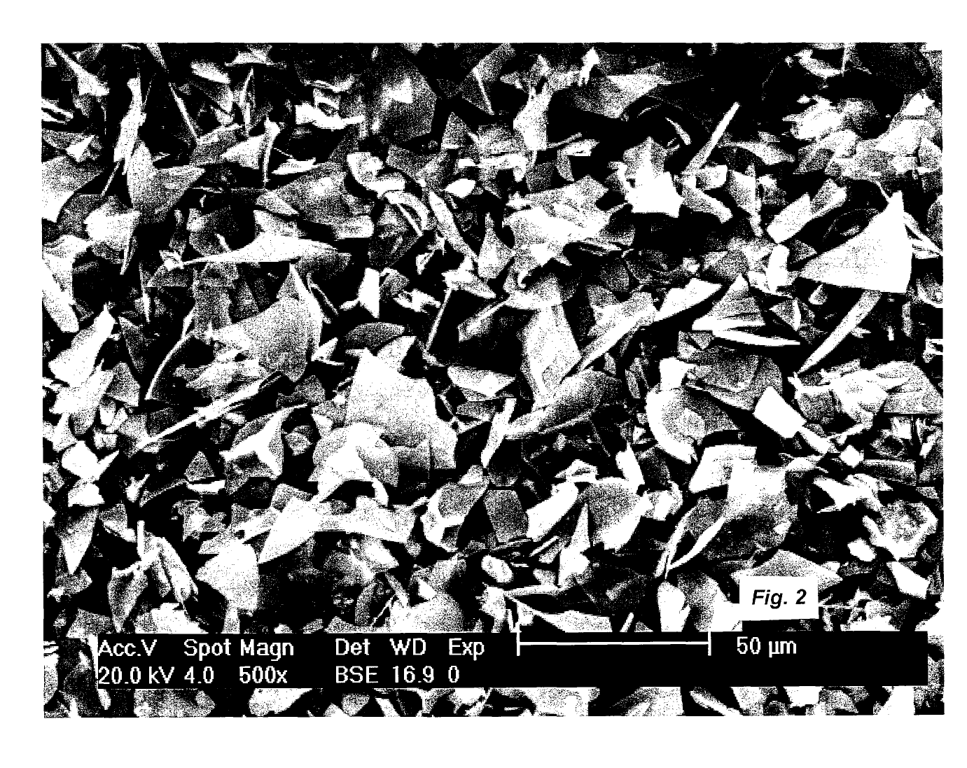

FIG. 1 shows an example of large particles of natural glass from a coarse fraction of a classified product, in particular, expanded perlite. As can be seen in FIG. 1, perlite particles having a size greater than about 50 μm tend to be generally three-dimensional, multi-angular particles. In contrast, as shown in FIG. 2 fine perlite particles from the fine fraction of the classified product having a size less than about 50 μm tend to be generally two-dimensional and relatively more platy than the larger particles. Thus, it is believed that natural glass particles (e.g., expanded perlite particles) having a size greater than about 50 μm tend to be more abrasive than particles having a smaller size. Further, it is also believed that smaller, platy particles tend to break down to even smaller particles more easily during, for example, a cleaning process.

According to some exemplary embodiments, natural glass, for example, commercially-available natural glass such as expanded perlite, may be milled and classified, such that the milled and classified natural glass has a top particle size (d₉₀) less than 50 μm. Dynamic classifiers (e.g., mechanical air classifiers), static classifiers (e.g., cyclones), and sieving can be used to control the top particle size of the expanded perlite. For example, an expanded perlite having a top particle size (d₉₀) of 112 μm, a median particle size (d₅₀) of 60 μm, and a (d₁₀) particle size of 22 μm may be milled and/or classified according to methods known to those skilled in art to obtain perlite having a top particle size (d₉₀) less than 50 μm. (A particle size designated “(d₁₀)” is defined as the size for which 10 percent of the volume of the particles is smaller than the indicated size.) For example, the milled and/or classified natural glass may have a top particle size (d₉₀) less than 45 μm, such as, for example, a top particle size (d₉₀) less than 40 μm or less than 30 μm. According to some embodiments, the natural glass has a top particle size (d₉₀) ranging from 20 μm to 40 μm, such as, for example, from 25 μm to 35 μm.

According to some exemplary embodiments, the milled and/or classified natural glass (e.g., expanded perlite) may have a median particle size (d₅₀) less than 30 μm. For example, the natural glass may have a median particle size (d₅₀) less than 25 μm, such as, for example, a median particle size (d₅₀) less than 20 μm. Some embodiments have a median particle size (d₅₀) ranging from 5 μm to 25 μm, such as, for example, from 10 μm to 20 μm.

FIG. 2 shows an example of an expanded perlite that has been milled and/or classified in the exemplary manner described above, and it shows the relatively two-dimensional and platy nature of the milled and/or classified perlite relative to the coarse perlite shown in FIG. 1. As explained in more detail herein, compositions for cleaning teeth that include natural glass with these exemplary particle size characteristics may result in compositions that result in effective cleaning of the teeth without adversely increasing the abrasiveness of the composition. It is believed that this may result from the relatively smaller natural glass particles having a relatively platy characteristic that increases the area of contact with the tooth relative to the sharp point-like contact of the three-dimensional and angular nature of relatively larger natural glass particles.

Some embodiments of compositions for cleaning teeth include natural glass in an amount ranging from, for example, 0.1 percent to 25 percent by weight of the weight of the composition, for example, from 0.1 percent to 15 percent by weight. According to some embodiments, natural glass may be present in an amount ranging from, for example, 0.1 percent to 10 percent by weight of the composition, or, for example, from 0.1 percent to 3 percent by weight.

According to some embodiments, a composition for cleaning teeth includes natural glass, wherein the natural glass is perlite. For example, according to some embodiments, the natural glass is expanded perlite. The perlite may be milled and classified, expanded perlite. According to some embodiments, the natural glass is unexpanded perlite.

Exemplary embodiments of compositions for cleaning teeth include natural glass and exhibit an RDA value less than 220. For example, some embodiments of compositions for cleaning teeth include natural glass and exhibit an RDA value less than 200, for example, less than 180. Some exemplary embodiments of compositions for cleaning teeth include natural glass and exhibit a PCR value of at least 110. For example, some embodiments include natural glass and exhibit a PCR value of at least 120.

Some embodiments of compositions for cleaning teeth are dentifrice compositions. According to some embodiments, the dentifrice composition is toothpaste, in particular, a dentifrice including a toothpaste base. For example, the toothpaste base may include at least one ingredient chosen from binders, such as thickening agents and/or gelling agents, humectants, foaming agents such as detergents, and polishing agents. The toothpaste base may also contain at least one additional ingredient chosen from, for example, water, preservative agents, flavoring agents, sweeteners, and fluoride containing compounds. It will be readily apparent to the skilled artisan that the components and their relative amounts in the toothpaste base may be modified to achieve the desired toothpaste product.

The toothpaste base according to some embodiments may contain at least one binder, such as thickeners, which may also be referred to as gelling agents. Any art-recognized gelling or thickening agent may be used. Thickening or gelling agents may be selected from natural, synthetic, and gum-like materials, including, but not limited to, carboxyl methyl cellulose, carrageenin, xantham gum, bentonite, and hydrated silica. The at least one thickening or gelling agent may be present in the toothpaste base in an amount ranging from, for example, about 0.1 percent to about 5 percent by weight, for example, from about 0.1 percent to about 3 percent by weight. According to some embodiments, the at least one thickening or gelling agent is present in the toothpaste base in an amount ranging from, for example, about 0.5 percent to about 1.5 percent by weight. Natural glass, such as classified expanded perlite having a smaller top particle size, can also be used as thickener.

According to some embodiments, the toothpaste base may also contain at least one ingredient chosen from detergents and surfactants. Suitable non-limiting examples of appropriate detergents for use in the toothpaste base include anionic surfactants, such as sodium alkylsulfates, sodium laurylsulfate, sodium myristylsulfate and sulfosuccinic acid surfactants; dialkyl sodium sulfosuccinate; non-anionic surfactants; and amphoteric surfactants. The at least one ingredient chosen from detergents and surfactants may be present in the toothpaste base in an amount ranging from, for example, about 0.1 percent to about 10 percent by weight, for example, from about 0.1 percent to about 5 percent by weight, and further, for example, from about 0.5 percent to about 3 percent by weight.

According to some embodiments, the toothpaste base may also contain at least one humectant, such as, for example, humectants chosen from glycerin, sorbitol, propylene glycols, polyethylene glycols, and mixtures thereof. The at least one humectant may be present in the toothpaste base in an amount ranging from, for example, about 10 percent to about 90 percent by weight, for example, from about 20 percent to about 80 percent by weight. According to some embodiments, the at least one humectant may be present in an amount ranging from about 30 percent to about 70 percent by weight.

Some embodiments of toothpaste base may contain at least one coloring or whitening agent. Any art-recognized coloring or whitening agent may be used. Coloring and whitening agents may include, for example, titanium dioxide. Coloring or whitening agents may be present in the toothpaste base in an amount ranging from about 0.1 percent to about 5 percent by weight, for example, ranging from about 0.1 percent to about 3 percent by weight, or, for example, ranging from about 0.1 percent to about 1 percent by weight.

The toothpaste base according to some embodiments may contain at least one preservative. Any art-recognized preservative may be used. For example, preservatives may be selected from sodium benzoate and methyl paraben. Preservatives may be present in the toothpaste base in an amount ranging from, for example, about 0.1 percent to about 3 percent, by weight, for example, ranging from about 0.1 percent to about 1 percent by weight, and further, for example, from about 0.1 percent to about 0.5 percent by weight. The toothpaste composition may further contain at least one additional ingredient chosen from therapeutic ingredients and preventatives such as water-insoluble non-cationic antibacterial agents, for example, triclosan, and cationic antibacterial agents.

The toothpaste base may also contain at least one foaming agent. Any art-recognized foaming agent may be used, and appropriate foaming agents will be readily apparent to the skilled artisan. Further, the toothpaste base may contain at least one flavoring agent. Any art-recognized flavoring agent may be used, and appropriate flavoring agents will be readily apparent to the skilled artisan. For example, flavoring agents may be chosen from oils of spearmint, peppermint, wintergreen, sassafras, clove, sage, eucalyptus, cinnamon, lemon, orange, and methyl salicylate.

The toothpaste base may contain at least one sweetener. Any art-recognized sweetener may be used, and appropriate sweeteners will be readily apparent to the skilled artisan. For example, sweeteners may be chosen from at least one of sucrose, lactose, maltose, xylitol, sodium cyclamate, perillartine, aspartyl phenyl alanine methyl ester, and saccharine.

The toothpaste base may contain fluoride, such as, any compatible composition that will dissociate and release fluorine-containing ions in water. Fluoride compositions may be chosen from one or more of sodium fluoride, stannous fluoride, sodium monofluorophosphate, potassium fluoride, potassium stannous fluoride, sodium fluorostannate, stannous chlorofluoride, and amine fluoride. Fluorides may be present in the toothpaste base in an amount ranging from about, for example, 0.1 percent to about 3 percent, by weight, for example, from about 0.1 percent to about 1 percent by weight, and further, for example, from about 0.2 percent to about 0.8 percent by weight.

Compositions according to some embodiments may also include abrasive materials chosen from any fluoride compatible abrasive material. Suitable non-limiting examples of abrasive materials that may be used may be chosen from, for example, natural glass, silica, alumina, aluminosilicate, dicalcium phosphate, sodium bicarbonate, sodium metaphosphate, potassium metaphosphate, tricalcium phosphate, calcium pyrophosphate, calcium carbonate, and bentonite. According to some embodiments, abrasives may be present in an amount ranging from about 4 percent to about 25 percent by weight, relative to the total weight of the composition.

### EXAMPLES

### Preparation of Examples

Three examples of natural glass were prepared and tested according to the following description. Examples 1-3 were prepared using a pilot scale Alpine™ 200 ATP air classifier (marketed by Hosokawa Alpine Aktiengesellchaft of Augsburg, Germany). Other dynamic classifiers (e.g., mechanical air classifiers) and other classification methods such as static classifiers (e.g., cyclones), and sieving (such as vibration screener and centrifugal sifter) known to those skilled in the art may be used. The mechanical air classifier used generally includes a horizontally mounted high speed classifying wheel and a classifying air outlet. Classifying air injected into the machine base flows inwards through the classifying wheel and discharges the fine particles, and coarse particles rejected by the classifying wheel are ejected from the classifier through a coarse material outlet. By adjusting operating parameters of the classifier, such as, for example, classifier wheel speed and air flow pressure, a material having the desired characteristics may be achieved.

Examples 1-3 were obtained from a commercially-available milled, expanded perlite product, Harborlite® 2000, which was used as the feed material for the exemplary classifier described above. The feed material had a median particle size (d₅₀) of 60 μm, and a particle size distribution (PSD) from 22 μm (d₁₀) to 112 μm (d₉₀).

The particle size distribution of samples was determined in accordance with the phenomenon of scattered light from a laser beam projected through a stream of particles. The amount and direction of light scattered by the particles is measured by an optical detector array and then analyzed by a microcomputer, which calculates the size distribution of the particles in the sample stream. For example, the particle size data may be obtained on a Leeds and Northrup Microtrac X100 laser particle size analyzer (marketed by Leeds and Northrup of North Wales, Pa.). This instrument is capable of determining particle size distribution over a particle size range from 0.12 μm to 704 μm.

The color of the natural glass was determined using Hunter scale “L,” “a,” and/or “b” color data collected on a Spectro/plus Spectrophotometer (Color and Appearance Technology, Inc., Princeton, N.J.). The L-value indicates the level of lightness or darkness, the a-value indicates the level of redness or greenness, and the b-value indicates the level of yellowness or blueness. Blue light brightness was calculated from the L-, a-, and b-value data. A krypton-filled incandescent lamp was used as the light source. The instrument was be calibrated according to the manufacturer's instructions using a highly polished black glass standard and a factory-calibrated white opal glass standard.

Operating parameters such as classifier rotor speed and air flow pressure were adjusted to the values shown in Table 1 below to achieve Examples 1-3.

TABLE 1

| | Rotor Speed | Feed rate | Primary | Secondary | Yield |
|---|---|---|---|---|---|
| Example | (rpm) | (kg/hr) | Air (m³/h) | Air (m³/h) | (%) |
| Example 1 | 5100 | 210 | 410 | 490 | 52 |
| Example 2 | 4700 | 201 | 420 | 500 | 60 |
| Example 3 | 3700 | 211 | 430 | 500 | 72 |

The particle size distribution and color characteristics of Examples 1-3 are shown Table 2 below.

TABLE 2

| | | | | | | | Blue Light |
|---|---|---|---|---|---|---|---|
| Example | d₁₀ | d₅₀ | d₉₀ | L | a | b | Brightness |
| Example 1 | 7.53 | 18.21 | 34.36 | 91.76 | −0.11 | 1.58 | 82.13 |
| Example 2 | 8.46 | 20.47 | 38.70 | 91.47 | −0.07 | 1.61 | 81.56 |
| Example 3 | 12.71 | 29.69 | 57.22 | 91.26 | −0.11 | 1.86 | 80.85 |

### Testing of Examples

1. Relative Dentin Abrasion Test on Dentifrices

The RDA value indicates the relative abrasion level of dentifrices. The RDA testing procedure used was the American Dental Association (ADA)-recommended procedure for determining dentifrice abrasivity. Dentin specimens were placed in a neutron flux under the controlled conditions outlined by the ADA. The specimens were then mounted in methylmethacrylate so they would fit in a V-8 cross-brushing machine. The specimens were brushed for a 1,500 stroke, precondition run using a slurry consisting of 10 grams of ADA reference material in 50 milliliters of a 0.5% carboxymethylcellulose (CMC) glycerine solution. The brushes used were those specified by the ADA, and brush tension was 150 grams.

Following a precondition run, the RDA test was performed using the above parameters (150 grams and 1,500 strokes) in a sandwich design in which each test material slurry (25 grams/40 milliliters of water) was flanked by the reference material slurries (10 grams/50 milliliters 0.5% CMC).

Samples of 1 milliliter were taken, weighed (0.01 grams) and added to 5 milliliters of scintillation cocktail. The samples were mixed well and immediately placed on the scintillation counter for radiation detection. Following counting, the net counter per minute (CPM) values were divided by the weight of the sample to calculate a net CPM/gram of slurry. The net CPM/gram of the pre- and post-ADA reference material for each test slurry was then calculated and averaged to use in the calculation for RDA for the test material. The ADA material was assigned a value of 100, and its ratio to the test material was calculated. The results of the RDA test are reported in Table 3 below. It is noteworthy to mention these RDA tests were not based on an actual toothpaste formulation, and the perlite loading percentage is significantly higher than would be used in a conventional toothpaste formulation. These test results provide relative RDA comparison between different natural glass (perlite in this case) products with different top particle sizes.

2. Stained Pellicle Removal—Pellicle Cleaning Ratio Test

The PCR value is an indication of the ability of dentifrices to remove stained pellicle (i.e., an indication of the cleaning ability of dentifrice formulations). Previous studies (J. Dent. Res., 61:1236, 1982) have indicated that the results of this test with dentifrice slurries compare favorably with those obtained in controlled clinical trials. Thus, the results of this test using dentifrice slurries may be considered to predict clinical findings with a reasonable degree of confidence.

During the PCR test, bovine, permanent, central incisors were cut to obtain enamel specimens measuring approximately 10 millimeters square. The enamel specimens were then embedded in an autopolymerizing methacrylate resin, so that only the enamel surfaces were exposed. The enamel surfaces were then smoothed and polished on a lapidary wheel and lightly etched to expedite stain accumulation and adherence. The specimens were then placed on a rotating rod in a 37° C. incubator, alternately exposing them to air and to a staining broth solution consisting of trypticase soy broth, tea, coffee, mucin, FeCl₃, and Sarcina lutea (bacteria). The staining broth was changed, and the specimens were rinsed daily for approximately seven days. After seven days, a darkly-stained pellicle film was apparent on the enamel surfaces. The specimens were then rinsed, allowed to air dry, and refrigerated until use.

For purposes of the PCR test, all dentifrice examples were tested using specimens prepared at the same time. The amount of in vitro stain was graded photometrically (i.e., via a Minolta C221, colorimeter) using only the L value of the LAB scale. The area of the specimens scored was a ¼ inch diameter circle in the center of the enamel specimen. Specimens with scores between 25 and 42 (with 25 being more darkly stained) were used. On the basis of these scores, the specimens were divided into groups of 16 specimens each, with each group having the same average baseline score.

The specimens were then mounted on a mechanical V-8 cross-brushing machine equipped with soft nylon-filament (Oral-B™ 40) toothbrushes. Tension on the enamel surface was adjusted to 150 grams. The dentifrice samples were used as slurries prepared by mixing 25 grams of dentifrice with 40 milliliters of deionized water. The ADA abrasion reference material (Ca₂P₂O₇) was prepared by mixing 10 grams of the reference material in 50 milliliters of a 0.5% CMC solution. The specimens were thereafter brushed for 800 strokes (i.e., for 4½ minutes). To minimize mechanical variables, one specimen per group was brushed on each of the eight brushing heads. Fresh slurries were made after being used to brush four specimens. Following brushing, specimens were rinsed, blotted dry, and scored again for stain, as previously described.

The difference between the pre- and post-brushing stain scores was determined, and the mean and standard error were calculated for the reference group. The cleaning ratio for the reference material group was assigned a value of 100. The mean decrement of the reference group was divided into 100 to obtain a constant value to multiply by each individual test decrement within the study. The individual cleaning ratio of each specimen was thereafter calculated by multiplying the decrement by the constant. The results of the PCR test are summarized in the Table 3 below.

TABLE 3

| | | RDA (Relative | PCR (Pellicle |
|---|---|---|---|
| Sample ID | d₉₀ | Dentin Abrasion) | Cleaning Ratio) |
| Example 1 | 34.36 | 172.12 ± 2.93 | 121.19 ± 6.76 |
| Example 2 | 38.70 | 200.54 ± 2.91 | 126.66 ± 7.47 |
| Example 3 | 57.22 | 261.55 ± 9.02 | 129.40 ± 8.29 |

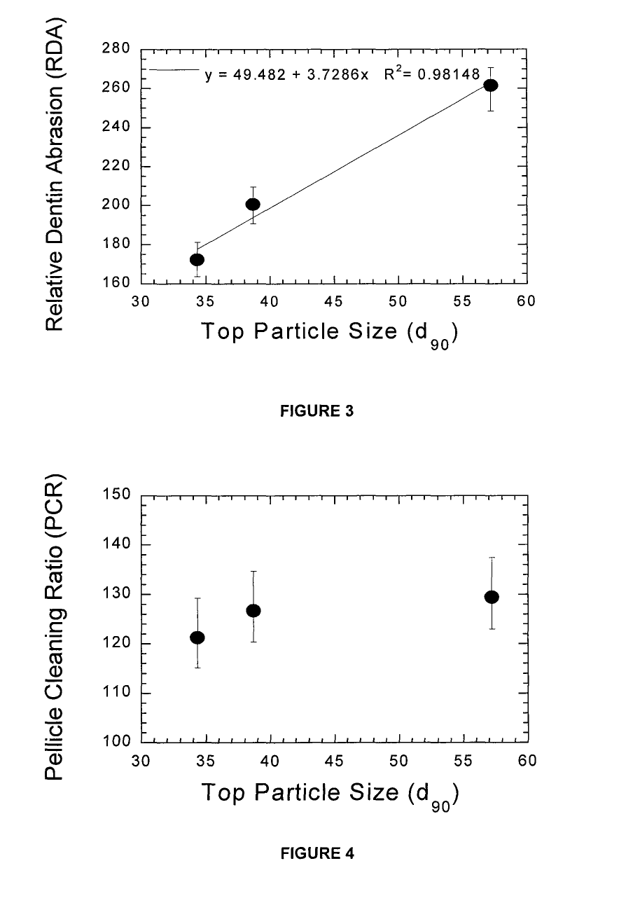

Table 3 and FIG. 3 show that abrasion decreases significantly with decreasing top particle size (d₉₀). For example, about a 30% reduction in abrasion can be achieved when top particle size (d₉₀) is less than 40 μm. As shown in FIG. 1, the perlite particles larger than 50 μm are generally three-dimensional and multi-angular in nature. Such particles tend to be more abrasive as compared to perlite particles that are less than 50 μm, which are generally two-dimensional and platy in nature. It is believed that platy particles tend easily break down during a cleaning process, which reduces abrasion. Table 3 also shows that the identified commercial baghouse perlite products have a top particle size (d₉₀) ranging from 57 μm to above 72 μm. It is believed that when particles 50 μm and larger are removed, abrasion can be significantly reduced.

Table 3 and FIG. 3 show that even with the smaller top particle size, Examples 1-3 are still effective in cleaning teeth. It is believed that this may be due to a greater contact area obtained with the relatively more platy surface of the smaller particles relative to the point-type contact with larger particles, which are relatively more three-dimensional and angular in character. Thus, compositions for cleaning teeth including natural glass having a smaller top particle size provide effective cleaning and reduced erosion of the teeth.

3. Polishing Effectiveness Test

The effectiveness of a milled and classified expanded perlite with smaller particle top size was measured using a polishing effectiveness test similar to that described in a paper by Bailey and Phillips (J. Dent. Res, 29:740, 1950) on the study of abrasive prophylactic agents and techniques on enamel surfaces.

The toothpaste samples for the polishing test contained sorbitol, water, perlite, hydrated silica (HCS—high cleaning silica), hydrated Silica (thickener), PEG, titanium dioxide, sodium lauryl sulfate, flavor, sodium saccharin, tetrasodium pyrophosphate, sodium fluoride, cellulose gum. Toothpaste sample 1 contained 20% high cleaning silica and Toothpaste sample 2 contained 2% perlite and 18% high cleaning silica. The perlite used in toothpaste sample 2 was a classified expanded perlite prepared by a method comparable to that of Example 1 above, and having a top particle size (d₉₀) of 35 microns.

Bovine permanent, central incisors were cut to obtain labial enamel specimens approximately 10×10 mm². The enamel specimens were embedded in an autopolymerizing methacrylate resin so that only the enamel surfaces were exposed. The enamel surfaces were smoothed and polished on a lapidary wheel. The bovine samples were prescreened by prophying them with a water slurry of LPA-3T abrasive (e.g., an aluminum oxide optical finishing powder) to a high luster.

The bovine samples were scored for surface reflectance using a beam reflectometer. Specimens were placed under the light source and the entire enamel labial surface was scanned observing the highest reading (the highest polished area). A polish score of at least 7.0 was achieved before any specimen is accepted for use in the study. This is based on a black onyx reference block being set on a reflectance value of 6.0. This procedure was used to confirm the specimens' ability to achieve a high polish.

The specimens were etched by decalcifying them in 1% HCL (v/v) for 2 minutes to provide a dull surface to initiate the study. The subsequent reflectometer baseline reading was around 2.0. The specimens were also scored using the Novo-Curve Glossmeter. The specimens were scored with the glossmeter after the reflectometer reading had been performed and then rotated 180 and scored again. The average of the two scores was used to calculate the glossmeter data.

Following the baseline (etched) scoring; the specimens were placed on a cross-brushing machine. The brush tension was adjusted to 150 grams, and the specimens were brushed for 4,500 strokes with the appropriate dentifrice slurry (25 grams of dentifrice+40 grams of deionized water, 10 grams of powder+50 ml 0.5% CMC) and a medium brush (Oral B-40). The specimens were removed from the brushing machine, rinsed and scored with both the reflectometer and the glossmeter once again for polish. Etching dulled the specimens again and the entire procedure was repeated additional times so that each product was assayed on each tooth set. The treatment design was a modified Latin Square design so that no treatment followed another treatment consistently.

The difference between the baseline score and the post-brushing score was calculated for each specimen and represents the polish increment. The mean, standard deviation and standard error of the polish increments was calculated for each group.

Statistical analyses were analyzed using a one-way analysis of variance model [Sigma Plot (11.0) Software]. Since significant differences were found, additional all pair wise comparisons were done using the Student-Newman-Keuls method. All analyses were done with the significance level set at below 0.05. The results are set forth in Table 4 below.

TABLE 4

| | High Cleaning | Perlite | Mean Polish Increment |
|---|---|---|---|
| Sample ID | Silica (%) | (%) | (Glossmeter) |
| Toothpaste sample 1 | 20 | 0 | 24.56 ± 4.23 |
| Toothpaste sample 2 | 18 | 2 | 37.24 ± 3.29 |

Table 4 shows that the natural glass having a smaller top particle size is more effective in tooth polishing compared to a hydrated silica control. The small platy perlite particles are thought to provide effective tooth cleaning and tooth polishing.

Perlite having a smaller top particle size can provide multiple functions in the toothpaste as an abrasive for cleaning and polishing and also as a thickener. Since it is a natural glass, it can be used in the all natural toothpaste formulations.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A composition for cleaning teeth, the composition comprising natural glass, wherein the natural glass has a top particle size (d₉₀) less than 50 μm and a median particle size (d₅₀) less than 30 μm, and wherein the natural glass ranges from 0.1 percent to 25 percent by weight of the composition.

2. The composition of claim 1, wherein the natural glass comprises perlite.

3. The composition of claim 1, wherein the natural glass comprises expanded perlite.

4. The composition of claim 1, wherein the natural glass comprises milled and classified, expanded perlite.

5. The composition of claim 1, wherein the natural glass comprises unexpanded perlite.

6. The composition of claim 1, wherein the natural glass is selected from pumice, obsidian, volcanic ash, and shirasu.

7. The composition of claim 1, wherein the natural glass has a top particle size (d₉₀) less than 40 μm.

8. The composition of claim 1, wherein the natural glass has a top particle size (d₉₀) less than 35 μm.

9. The composition of claim 1, wherein the natural glass has a top particle size (d₉₀) ranging from 20 μm to 40 μm.

10. The composition of claim 1, wherein the natural glass has a top particle size (d₉₀) ranging from 25 μm to 35 μm.

11. The composition of claim 1, wherein the natural glass has a median particle size (d₅₀) less than 25 μm.

12. The composition of claim 1, wherein the natural glass has a median particle size (d₅₀) ranging from 5 μm to 25 μm.

13. The composition of claim 1, wherein the natural glass has a median particle size (d₅₀) ranging from 10 μm to 20 μm.

14. The composition of claim 1, wherein the natural glass ranges from 0.1 percent to 15 percent by weight of the composition.

15. The composition of claim 1, wherein the natural glass ranges from 0.1 percent to 10 percent by weight of the composition.

16. The composition of claim 1, wherein the natural glass ranges from 0.1 percent to 5 percent by weight of the composition.

17. The composition of claim 1, wherein the natural glass ranges from 0.1 percent to 3 percent by weight of the composition.

18. The composition of claim 1, wherein the composition exhibits an RDA value less than 200.

19. The composition of claim 1, wherein the composition exhibits an RDA value less than 150.

20. The composition of claim 1, wherein the composition exhibits an RDA value less than 100.

21. The composition of claim 1, wherein the composition exhibits an RDA value less than 80.

22. The composition of claim 1, wherein the composition exhibits a PCR value of at least 80.

23. The composition of claim 1, wherein the composition exhibits a PCR value of at least 100.

24. The composition of claim 1, wherein the composition exhibits a PCR value of at least 120.

25. The composition of claim 1, wherein the composition exhibits a PCR value of at least 150.

26. The composition of claim 1, further comprising a toothpaste base.

27. The composition of claim 26, wherein the toothpaste base comprises at least one of binders, humectants, abrasives, detergents, flavoring agents, and preventatives.

28. A natural glass composition, wherein the natural glass has a top particle size (d₉₀) less than 50 μm, a median particle size (d₅₀) less than 30 μm, and exhibits an RDA value less than 200.

29. The composition of claim 28, wherein the natural glass comprises perlite.

30. The composition of claim 28, wherein the natural glass comprises expanded perlite.

31. The composition of claim 28, wherein the natural glass comprises milled, expanded perlite.

32. The composition of claim 28, wherein the natural glass comprises unexpanded perlite.

33. The composition of claim 28, wherein the natural glass has a top particle size (d₉₀) less than 40 μm.

34. The composition of claim 28, wherein the natural glass has a top particle size (d₉₀) less than 35 μm.

35. The composition of claim 28, wherein the natural glass has a top particle size (d₉₀) ranging from 20 μm to 40 μm.

36. The composition of claim 28, wherein the natural glass has a top particle size (d₉₀) ranging from 25 μm to 35 μm.

37. The composition of claim 28, wherein the natural glass has a median particle size (d₅₀) less than 25 μm.

38. The composition of claim 28, wherein the natural glass has a median particle size (d₅₀) ranging from 5 μm to 25 μm.

39. The composition of claim 28, wherein the natural glass has a median particle size (d₅₀) ranging from 10 μm to 20 μm.

40. The composition of claim 28, wherein the composition exhibits an RDA value less than 200.

41. The composition of claim 28, wherein the composition exhibits an RDA value less than 150.

42. The composition of claim 28, wherein the composition exhibits a PCR value of at least 80.

43. The composition of claim 28, wherein the composition exhibits a PCR value of at least 100.

44. The composition of claim 28, wherein the composition exhibits a PCR value of at least 120.

45. The composition of claim 28, further comprising a toothpaste base.

46. The composition of claim 45, wherein the toothpaste base comprises at least one of binders, humectants, abrasives, detergents, flavoring agents, and preventatives.

47. The composition of claim 28, wherein the natural glass is selected from pumice, obsidian, volcanic ash, and shirasu.
